(19) United States
(12) Patent Application Publication
(10) Pub. No.: US 2020/0217838 A1
(43) Pub. Date: Jul. 9, 2020

(54) DEVELOPMENT OF BIOMARKER FOR KETOGENIC DIET-BASED CANCER TREATMENT

(71) Applicants: OSAKA UNIVERSITY, Osaka (JP); MEIJI CO., LTD., Tokyo (JP)

(72) Inventors: Keisuke HAGIHARA, Osaka (JP); Kinya ASHIDA, Kanagawa (JP); Kentaro NAKAMURA, Kanagawa (JP)

(73) Assignees: OSAKA UNIVERSITY, Osaka (JP); MEIJI CO., LTD., Tokyo (JP)

(21) Appl. No.: 16/648,708

(22) PCT Filed: Sep. 21, 2018
(86) PCT No.: PCT/JP2018/035043

§ 371 Date: Mar. 19, 2020

(30) Foreign Application Priority Data
Sep. 22, 2017 (JP) 2017-182683

(51) Int. Cl. G01N 33/50; G01N 33/574

(52) U.S. Cl. CPC G01N 33/5091; G01N 33/574

(57) ABSTRACT

A method for selecting a cancer patient for whom a ketogenic diet therapy would be effective and a method for predicting efficacy of a ketogenic diet therapy in a cancer patient are provided. A method is characterized by obtaining a result of analysis of at least one pre-intake candidate substance selected from the group consisting of acylcarnitine(12:0), acylcarnitine(12:1)-2, acylcarnitine(12:1)-3, acylcarnitine(14:1)-1, acylcarnitine(14:2)-1, acylcarnitine(14:2)-2, acylcarnitine(14:3)-2, acylcarnitine(16:1), acylcarnitine(16:2), anandamide, heptadecenoic acid, hypotaurine, linoleylethanolamide, palmitoleic acid, palmitoylcarnitine, phytosphingosine, quinic acid, uric acid, alanine, citrulline, tryptophan and lysine in a serum derived from a cancer patient prior to intake of a ketogenic diet, and using the result of analysis as an indicator.

### TECHNICAL FIELD

The present invention relates to development of biomarkers for cancer ketogenic diet therapies. Specifically, the present invention relates to a method for selecting a cancer patient for whom a ketogenic diet therapy would be effective and a method for predicting efficacy of a ketogenic diet therapy in a cancer patient.

### BACKGROUND ART

The “ketogenic diet” is known as one of diet therapies. The “ketogenic diet” is a carbohydrate-restricted high-fat diet in which 60 to 90% of the energy intake is derived from fat. Therefore, the “ketogenic diet” is adopted as a diet for patients requiring a carbohydrate-restricted diet, for example, for treating epilepsy in childhood (for example, Patent Literature 1).

In recent years, it has been proposed that diet therapies adopting this “ketogenic diet” can be a therapy for cancer patients (see, Patent Literature 2). Cases in which dramatic clinical effects were observed by the ketogenic diet therapies have been reported. However, the efficacy of the ketogenic diet therapies has not been necessarily confirmed for all cancer patients and the details of the mechanism of action of the effect of the ketogenic diets in cancer patients are not known. As an approach for elucidation of the mechanism of action, searches for therapeutic markers and response prediction markers have been required.

### CITATION LIST

### Patent Literature

Patent Literature 1: Japanese Patent No. 5937771
Patent Literature 2: International Publication No. WO 2017/038101

### SUMMARY OF INVENTION

### Technical Problem

Ketogenic diets impose not a little burden on cancer patients, since ketogenic diets are high lipid diets. To reduce the burden on cancer patients, it is required to select a cancer patient(s) for whom ketogenic diets would be effective with a high probability. In addition, when a ketogenic diet therapy has already been started, predicting, if possible, whether the effect of the ketogenic diet therapy will be achieved or not before its completion will lead to the reduction of the burden on the patient(s).

Accordingly, an object of the present invention is to provide a method for selection that enables easy selection of a cancer patient for whom a ketogenic diet therapy would be effective before starting the ketogenic diet therapy. Moreover, an object of the present invention is to provide a method for predicting an efficacy of a ketogenic diet therapy prior to completing the ketogenic diet therapy on a cancer patient.

### Solution to Problem

The method for selecting a cancer patient for whom a ketogenic diet therapy would be effective according to the present invention comprises obtaining a result of analysis of at least one pre-intake candidate substance selected from the group consisting of acylcarnitine(12:0), acylcarnitine(12:1)-2, acylcarnitine(12:1)-3, acylcarnitine(14:1)-1, acylcarnitine(14:2)-1, acylcarnitine(14:2)-2, acylcarnitine(14:3)-2, acylcarnitine(16:1), acylcarnitine(16:2), anandamide, heptadecenoic acid, hypotaurine, linoleylethanolamide, palmitoleic acid, palmitoylcarnitine, phytosphingosine, quinic acid, uric acid, alanine, citrulline, tryptophan and lysine in a serum derived from a cancer patient prior to intake of a ketogenic diet, and using the result of analysis as an indicator.

The method for predicting an efficacy of a ketogenic diet therapy on a cancer patient according to the present invention comprises obtaining a result of analysis of at least one post-intake candidate substance selected from the group consisting of diethanolamine, histidine, hydroxyproline, stearoylethanolamide, stigmasterol, cystine and tyrosine in a serum derived from a cancer patient who has taken a ketogenic diet, and using the result of analysis as an indicator.

### Effects of Invention

According to the method for selection according to the present invention, a result of analysis of at least one of pre-intake candidate substances in a serum derived from a cancer patient prior to intake of a ketogenic diet is obtained. By using the result of analysis thus obtained as an indicator, a cancer patient(s) for whom the ketogenic diet therapy would be effective can be selected prior to intake of the ketogenic diet.

Moreover, according to the method for prediction according to the present invention, a result of analysis of at least one of post-intake candidate substances in a serum derived from a cancer patient who has taken a ketogenic diet is obtained. By using the result of analysis thus obtained as an indicator, the efficacy of the ketogenic diet therapy on the cancer patient can be predicted prior to completing the ketogenic diet therapy.

### BRIEF DESCRIPTION OF DRAWINGS

FIG. 1 illustrates a result of a partial least squares (PLS) analysis conducted using a result of a metabolome analysis.

### DESCRIPTION OF EMBODIMENTS

Embodiments of the present invention will be described in detail below.

In the present invention, the ketogenic diet refers to a diet having a ketone ratio (lipid:(protein+carbohydrate)) in a range of from about 2:1 to 1:1 in weight ratio. The ketogenic diet therapy is not particularly limited as to the other nutrients as long as such a ketogenic diet is taken for a predetermined period of time (for example, 3 months). During the ketogenic diet therapy, required trace elements and vitamins may be taken in by using a supplement(s) or the like as appropriate.

Examples of the “cancer” as used herein include tumors that have developed as a result of mutation of normal cells. A malignant tumor may occur in any organ or tissue in the whole body. Examples of the cancer patients herein include patients suffering from cancers such as lung cancer, ovarian cancer, bladder cancer, lip adenoid cystic carcinoma, kidney cancer, urothelial cancer, colorectal cancer, prostate cancer, glioblastoma multiforme, pancreatic cancer, breast cancer, melanoma, liver cancer, gastric cancer and esophageal cancer. The cancer patients are not particularly limited as to sex and age, as long as they can take a ketogenic diet, and any cancer patient may be treated by the present invention.

In the method for selection according to the present invention, a serum collected from a cancer patient prior to intake of a ketogenic diet is used as a specimen. A cancer patient(s) for whom the ketogenic diet therapy would be effective is selected using, as an indicator, the result of analysis of at least one of the pre-determined pre-intake candidate substances among the substances contained in the specimen. The pre-intake candidate substances are acylcarnitine(12:0), acylcarnitine(12:1)-2, acylcarnitine(12:1)-3, acylcarnitine(14:1)-1, acylcarnitine(14:2)-1, acylcarnitine(14:2)-2, acylcarnitine(14:3)-2, acylcarnitine(16:1), acylcarnitine(16:2), anandamide, heptadecenoic acid, hypotaurine, linoleylethanolamide, palmitoleic acid, palmitoylcarnitine, phytosphingosine, quinic acid, uric acid, alanine, citrulline, tryptophan and lysine.

The specimen can be analyzed, for example, by the metabolome analysis described below. It is preferred that the pre-intake candidate substances in the specimen are individually analyzed by, for example, the amino acid assay or the like using a high performance liquid chromatography (HPLC). A cancer patient(s) for whom the ketogenic diet therapy would be effective can be selected by using a result of analysis of at least one pre-intake candidate substance as an indicator. The greater is the number of the pre-intake candidate substances, the higher will be the precision of the selection of the cancer patient(s) for whom a ketogenic diet therapy would be effective.

In the method of prediction according to the present invention, a serum collected from a cancer patient who has taken a ketogenic diet is used as a specimen. The “cancer patient who has taken a ketogenic diet” refers to a cancer patient who is conducting a ketogenic diet therapy prior to completing the ketogenic diet therapy. The efficacy of the ketogenic diet therapy is predicted using, as an indicator, the result of analysis of at least one of pre-determined post-intake candidate substances among the substances contained in a specimen. The post-intake candidate substances are diethanolamine, histidine, hydroxyproline, stearoylethanolamide, stigmasterol, cystine and tyrosine.

It is preferred that the post-intake candidate substances in the specimen are analyzed by the amino acid assay or the like using HPLC, as described above, to obtain a result of the analysis. The efficacy of the ketogenic diet therapy can be predicted using the result of analysis of at least one post-intake candidate substance as an indicator. The greater is the number of the post-intake candidate substances, the higher will be the precision of the prediction of efficacy of the ketogenic diet therapy for the cancer patient.

The pre-intake candidate substances and the post-intake candidate substances can be determined based on the results of the ketogenic diet therapy applied to a group of test patients. The test patients refer to patients suffering from a cancer as described above. The test patients are not particularly limited as to sex and age, as long as they can take a ketogenic diet.

The pre-intake candidate substances can be determined by the method described below. First, sera derived from a group of test patients prior to intake of the ketogenic diet are analyzed by a metabolome analysis to obtain results of analysis of a large number of substances in the sera. For the metabolome analysis, for example, capillary electrophoresis time-of-flight mass spectrometry (CE-TOF/MS) and liquid chromatography time-of-flight mass spectrometry (LC-TOF/MS) can be used.

Peaks detected for respective substances are annotated referring to a metabolite library and a statistical analysis is conducted to obtain a result of analysis. Usually, about 200 kinds of substances in a serum can be analyzed by a metabolome analysis. The results of analysis of each of the substances in the serum derived from each test patient are recorded as “results of analysis prior to intake of the ketogenic diet”.

A group of test patients is fed with a predetermined ketogenic diet to start a ketogenic diet therapy. Three months later, therapeutic effects on the test patients who can be clinically evaluated are evaluated. The “test patients who can be clinically evaluated” refers to “test patients having taken the ketogenic diet for at least 3 months”. The therapeutic effects are evaluated based on the size of tumor in CT images as follows.

Complete Remission (CR): Complete disappearance of tumor

Progressive Disease (PD): An increase of 20% or more in the sum of tumor sizes and an increase in absolute size of 5 mm or more, or appearance of new lesions

Stable Disease (SD): No change in tumor size

Test patients evaluated as complete remission are included in a remission group (hereinafter, also referred to as CR group), test patients evaluated as progressive disease and test patients evaluated as stable disease are both included in a progressive disease/stable disease group (hereinafter, also referred to as PD/SD group). The recorded “results of analysis prior to the intake of the ketogenic diet” are classified into results of the CR group and results of the PD/SD group.

The results of the CR group and the results of the PD/SD group are compared for each substance and the significant difference between the both groups is determined by Welch's t test. Substances found to have a significant difference are extracted as pre-intake candidate substances.

The post-intake candidate substances can be determined by the method described below. Therapeutic effects on the test patients who had a predetermined ketogenic diet for 3 months and can be clinically evaluated are evaluated as described above. Based on the results of evaluation of the therapeutic effects, patients in the CR group and those in the PD/SD group are selected from the test patients as described above.

Sera derived from the patients in the CR group and those in the PD/SD group are analyzed by a metabolome analysis to obtain results of analysis of a large number of substances in the sera. The results of analysis of each substance in the sera will serve as the “results of analysis after intake of the ketogenic diet”. The “results of analysis after intake of the ketogenic diet” are compared between the CR group and the PD/SD group for each substance and a significant difference between the groups is judged as described above.

Among a large number of substances, the substances found to have a significant difference between the CR group and the PD/SD group are picked up as post-intake candidate substances.

### Action and Effect

In the method for selection according to this embodiment, predetermined substances in serum are used as pre-intake candidate substances. A cancer patient(s) for whom a ketogenic diet therapy would be effective can be selected prior to intake of the ketogenic diet by using, as an indicator, the result of analysis of at least one of pre-intake candidate substances in a serum(s) derived from a cancer patient(s) prior to intake of the ketogenic diet.

In the method of prediction according to this embodiment, predetermined substances different from the pre-intake candidate substances are used as post-intake candidate substances. The efficacy of a ketogenic diet therapy on a cancer patient can be predicted using, as an indicator, the result of analysis of at least one of the post-intake candidate substances in a serum derived from the cancer patient who has taken the ketogenic diet.

### EXAMPLES

The present invention will be more specifically described by Examples below, but the present invention is not limited by these Examples.

The number of patients in a test patient group was 5 (2 females and 3 males). As to the background of the patients, both females had lung cancer, among which one patient was 56 years old, 159 cm tall and 49.8 kg in weight and the other patient was 53 years old, 151 cm tall and 44.3 kg in weight, and among the three males, one had lung cancer, one had bladder cancer, and one had lip adenoid cystic carcinoma, and they were 60.6±13.0 years old, 174.1±0.23 cm tall, 61.5±5.8 kg in weight, and had BMI of 20.3±1.9.

Five serum specimens collected from the test patient group prior to intake of a ketogenic diet were analyzed by a metabolome analysis using CE-TOF/MS and LC-TOF/MS as described above and a large number of substances in the sera were statistically analyzed. The analyzers used were Agilient CE-TOFMS system (manufactured by Agilient Technologies, Inc.), Capillary: Fused silica capillary i. d. 50 μm×80 cm for CE-TOF/MS and LC system: Agilient 1200 series RPLC system SL (manufactured by Agilient Technologies, Inc.), Column: ODS Column 2×50 mm, 2 μm, and MS system: Agilient LC/MSD TOF (manufactured by Agilient Technologies, Inc.) for LC-TOF/MS.

In the analysis by CE-TOF/MS, a total of 173 peaks were detected (among them 108 in the cationic mode and 65 in the anionic mode). In the analysis by LC-TOF/MS, a total of 169 peaks were detected (among them 80 in the positive mode and 89 in the negative mode). The results of analysis of each substance in sera derived from the test patients were recorded as “results of analysis prior to intake of the ketogenic diet”.

The substances analyzed prior to the intake of the ketogenic diet included acylcarnitine(12:0), acylcarnitine(12:1)-2, acylcarnitine(12:1)-3, acylcarnitine(14:1)-1, acylcarnitine(14:2)-1, acylcarnitine(14:2)-2, acylcarnitine(14:3)-2, acylcarnitine 16:1), acylcarnitine(16:2), anandamide, heptadecenoic acid, hypotaurine, linoleoylethanolamide, palmitoleic acid, palmitoylcarnitine, phytosphingosine, quinic acid, uric acid, and the like.

The test patient group had a ketogenic diet therapy as described below. This clinical study was conducted with the approval of the Osaka University genome committee.

(1) During the first week, the calorie was 30 kcal/kg based on the net body weight, lipid was not restricted, protein was not restricted, and the targeted amount of carbohydrate was 10 g or less. Specifically, when the net body weight of a patient was, for example, 50 kg, the daily calorie was 1500 kcal, lipid was 140 g, protein was 60 g, and carbohydrate was 10 g.

The targeted ketone ratio (lipid: (protein+carbohydrate)) in the ketogenic diet was 2:1. Intake of the other nutrients was not restricted and a supplement or the like was used to take required trace elements and vitamins as appropriate.

(2) From the second week to the third month, the content of the diet was determined with reference to the blood level of ketone bodies. For the blood level of ketone bodies, the concentrations of acetoacetic acid and β-hydroxybutyric acid were measured. The daily intake level of carbohydrate was 20 g or less, the daily calorie level was 1400 to 1600 kcal, the daily lipid level was 120 to 140 g, the daily protein level was 70 g, the daily carbohydrate level was 20 g, and the targeted ketone ratio was 2:1 to 1:1. For supplementation of the calorie, “MCT oil” (manufactured by The Nisshin OilliO Group, Ltd.) or “Ketone formula” (manufactured by Meiji Co., Ltd.) was used.

At the start of the ketogenic diet therapy, the test patient group was given an explanation that temporary hypoglycemia, nausea, lassitude or the like may appear and an actual nutritional instruction was provided under supervision of a nutritionist.

Five serum specimens derived from the test patient group having taken the ketogenic diet for 3 months were analyzed by a metabolome analysis as described above and a large number of substances in the sera were statistically analyzed. The results of analysis of each of the substances in the specimens were recorded as “results of analysis after intake of the ketogenic diet” for each of the test patients.

The substances analyzed after the intake of the ketogenic diet included diethanolamine, histidine, hydroxyproline, stearoylethanolamide, stigmasterol and the like.

Furthermore, the therapeutic effects of the ketogenic diet therapy on the group of the test patients who have been subjected to the ketogenic diet for 3 months were evaluated. The therapeutic effects were classified into complete remission (CR), progressive disease (PD), and stable disease (SD) based on the criteria as described above.

Complete response group (CR group) and progressive disease/stable disease group (PD/SD group) were as follows.

CR group: 2 patients (2 females, both having lung cancer, one patient was 56 years old, 159 cm tall, and 49.8 kg in weight and the other patient was 53 years old, 151 cm tall, and 44.3 kg in weight)
PD/SD group: 3 patients (3 males, one patient having lung cancer, another patient having bladder cancer, and the other patient having lip adenoid cystic carcinoma, they were 50.6±13.0 years old, 174.1±0.23 cm tall, 61.5±5.8 kg in weight, and BMI: 20.3±1.9)

The recorded “results of analysis prior to the intake of the ketogenic diet” were classified into the CR group (2 specimens) and the PD/SD group (3 specimens), and the mean value and the standard deviation of peak areas were calculated for each substance. Furthermore, the peak area ratio (CR/(PD/SD)) was calculated for each substance and the significant difference was determined by the p value. Determination of the significant difference was effected by Welch's t test. The substances found to have a significant difference between the CR group and the PD/SD group are summarized in Table 1 below together with the calculation results.

The “results of analysis after intake of the ketogenic diet” were also classified into the CR group (2 specimens) and the PD/SD group (3 specimens), and the mean value and the standard deviation of peak area were calculated for each substance. Furthermore, the peak area ratio (CR/(PD/SD)) was calculated for each substance and a significant difference was evaluated by the p value. The substances found to have a significant difference between the CR group and the PD/SD group are summarized in Table 2 below together with the calculation results.

TABLE 1

| | CR | | PD/SD | | CR/ | |
|---|---|---|---|---|---|---|
| Name of | Mean | Standard | Mean | Standard | (PD/ | p |
| substance | value | deviation | value | deviation | SD) | value |
| Acylcarnitine | 9.8E−06 | 6.8E−06 | 4.0E−05 | 1.1E−05 | 0.2 | 0.031 |
| (12:0) | | | | | | |
| Acylcarnitine | 3.4E−06 | 1.0E−06 | 1.5E−05 | 9.9E−07 | 0.2 | 0.004 |
| (12:1)-2 | | | | | | |
| Acylcarnitine | 4.1E−06 | 4.4E−07 | 1.9E−05 | 5.5E−06 | 0.2 | 0.040 |
| (12:1)-3 | | | | | | |
| Acylcarnitine | 1.9E−05 | 6.3E−06 | 9.7E−05 | 1.3E−05 | 0.2 | 0.004 |
| (14:1)-1 | | | | | | |
| Acylcarnitine | 2.4E−06 | 5.4E−07 | 9.4E−06 | 1.9E−06 | 0.3 | 0.017 |
| (14:2)-1 | | | | | | |
| Acylcarnitine | 7.0E−06 | 3.3E−06 | 4.0E−05 | 4.5E−06 | 0.2 | 0.003 |
| (14:2)-2 | | | | | | |
| Acylcarnitine | 1.1E−06 | 5.5E−07 | 5.4E−06 | 1.2E−06 | 0.2 | 0.013 |
| (14:3)-2 | | | | | | |
| Acylcarnitine | 1.8E−05 | 3.3E−06 | 3.9E−05 | 4.3E−06 | 0.5 | 0.011 |
| (16:1) | | | | | | |
| Acylcarnitine | 1.9E−06 | 7.3E−07 | 7.7E−06 | 8.0E−07 | 0.3 | 0.007 |
| (16:2) | | | | | | |
| Anandamide | 2.5E−06 | 1.4E−08 | 2.0E−06 | 1.3E−07 | 1.2 | 0.022 |
| Heptadecenoic acid | 5.6E−06 | 4.6E−07 | 1.4E−05 | 2.6E−06 | 0.4 | 0.025 |
| Hypotaurine | 3.4E−04 | 1.1E−05 | 5.6E−04 | 5.5E−05 | 0.6 | 0.015 |
| Linoleyl- | 3.8E−05 | 1.5E−07 | 3.0E−05 | 2.1E−06 | 1.3 | 0.022 |
| ethanolamide | | | | | | |
| Palmitoleic acid | 4.7E−05 | 1.3E−05 | 1.3E−04 | 2.9E−05 | 0.3 | 0.022 |
| Palmitoylcamitine | 7.1E−05 | 7.8E−06 | 1.4E−04 | 1.4E−05 | 0.5 | 0.005 |
| Phytosphingosine | 5.5E−06 | 4.0E−07 | 8.6E−06 | 6.9E−07 | 0.6 | 0.008 |
| Quinic acid | 9.4E−04 | 9.7E−05 | 2.5E−04 | 9.4E−05 | 3.8 | 0.012 |
| Uric acid | 2.2E−02 | 1.9E−03 | 3.2E−02 | 2.6E−03 | 0.7 | 0.016 |

TABLE 2

| Name | CR | | PD/SD | | CR/ | |
|---|---|---|---|---|---|---|
| of | Mean | Standard | Mean | Standard | (PD/ | p |
| substance | value | deviation | value | deviation | SD) | value |
| Dietha- | 7.0E−04 | 1.2E−05 | 2.8E−04 | 2.5E−05 | 2.5 | 0.009 |
| nolamine | | | | | | |
| Histidine | 3.1E−02 | 1.0E−03 | 2.1E−02 | 8.2E−04 | 1.5 | 0.009 |
| Hydroxy- | 4.4E−03 | 3.3E−04 | 2.3E−03 | 2.2E−04 | 1.9 | 0.025 |
| proline | | | | | | |
| Stearoyl- | 1.1E−05 | 3.4E−07 | 8.3E−06 | 6.0E−07 | 1.3 | 0.048 |
| ethanol- | | | | | | |
| amide | | | | | | |
| Stigmas- | 7.0E−06 | 3.1E−07 | 3.8E−06 | 3.5E−07 | 1.8 | 0.011 |
| terol | | | | | | |

In Table 1 and Table 2 above, it is indicated that the results of analysis of substances in serum are different between the CR group and the PD/SD group. According to Table 1 above, the substances found to have a significant difference between the CR group and the PD/SD group before the intake of the ketogenic diet are acylcarnitine(12:0), acylcarnitine(12:1)-2, acylcarnitine(12:1)-3, acylcarnitine(14:1)-1, acylcarnitine(14:2)-1, acylcarnitine(14:2)-2, acylcarnitine(14:3)-2, acylcarnitine(16:1), acylcarnitine(16:2), anandamide, heptadecenoic acid, hypotaurine, linoleylethanolamide, palmitoleic acid, palmitoylcarnitine, phytosphingosine, quinic acid and uric acid.

Acylcarnitine(12:0), acylcarnitine(12:1)-2, acylcarnitine(12:1)-3, acylcarnitine(14:1)-1, acylcarnitine(14:2)-1, acylcarnitine(14:2)-2, acylcarnitine(14:3)-2, acylcarnitine(16:1), acylcarnitine(16:2), anandamide, heptadecenoic acid, hypotaurine, linoleylethanolamide, palmitoleic acid, palmitoylcarnitine, phytosphingosine, quinic acid and uric acid in serum prior to the intake of a ketogenic diet will serve as an indicator for determining whether the ketogenic diet therapy will be effective for a patient or not. In other words, these 18 substances are pre-intake candidate substances.

Therefore, a cancer patient for whom the ketogenic diet therapy would be effective can be selected by analyzing a serum derived from a cancer patient prior to the intake of the ketogenic diet and using, as an indicator, a result of analysis of at least one of acylcarnitine(12:0), acylcarnitine(12:1)-2, acylcarnitine(12:1)-3, acylcarnitine(14:1)-1, acylcarnitine(14:2)-1, acylcarnitine(14:2)-2, acylcarnitine(14:3)-2, acylcarnitine(16:1), acylcarnitine(16:2), anandamide, heptadecenoic acid, hypotaurine, linoleylethanolamide, palmitoleic acid, palmitoylcarnitine, phytosphingosine, quinic acid and uric acid.

Moreover, according to Table 2 above, the substances found to have a significant difference between the CR group and the PD/SD group after the intake of the ketogenic diet for 3 months are diethanolamine, histidine, hydroxyproline, stearoylethanolamide and stigmasterol.

Diethanolamine, histidine, hydroxyproline, stearoylethanolamide and stigmasterol in serum after intake of a ketogenic diet will serve as an indicator for determining whether the ketogenic diet therapy has effectively acted or not. In other words, these 5 substances are post-intake candidate substances.

Therefore, an efficacy of a ketogenic diet therapy on a cancer patient can be predicted by analyzing the serum derived from the cancer patient having taken the ketogenic diet and using, as an indicator, a result of analysis of at least one of diethanolamine, histidine, hydroxyproline, stearoylethanolamide and stigmasterol.

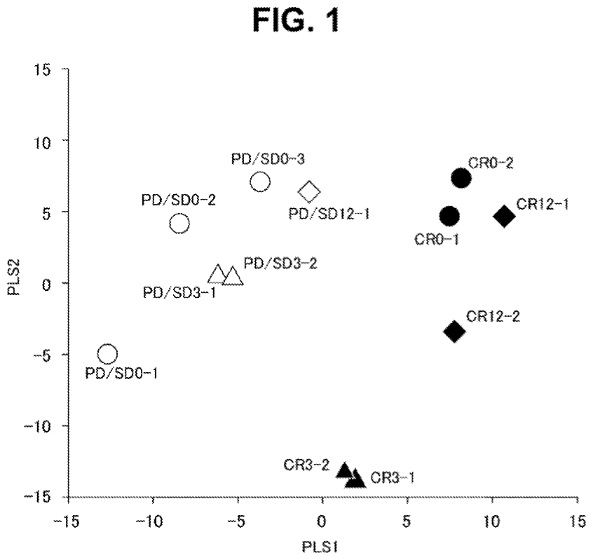

In addition, 2 test patients in the CR group and 1 test patient in the PD/SD group continued the ketogenic diet therapy for 12 months. Sera derived from the patients were also analyzed by the metabolome analysis as described above. The data thus obtained was combined with the above-mentioned data obtained prior to the start of the therapy and the data obtained after 3 months' therapy. Then a principal component analysis and a PLS analysis that gave group information were conducted. The results are illustrated in FIG. 1.

In FIG. 1, the filled circles (CR0-1, CR0-2) represent specimens from the CR group prior to the start, the filled triangles (CR3-1, CR3-2) represent specimens from the CR group 3 months later, the filled diamonds (CR12-1, CR12-2) represent specimens from the CR group 12 months later, the open circles (PD/SD0-1, PD/SD0-2, PD/SD0-3) represent specimens from the PD/SD group prior to the start, open triangles (PD/SD3-1, PD/SD3-2) represent specimens from the PD/SD group 3 months later, and the open diamond (PD/SD12-1) represents a specimen from the PD/SD group 12 months later.

Since the results of the specimens from the CR group and those from the PD/SD group are separated in the PLS1 axis direction, it was found that there is a correlation between the PLS1 axis and the group of specimens. By examining the factor loading corresponding to the PLS1 axis, substances having a high correlation can be extracted. The substances having a high correlation with the PLS1 axis were quinic acid, caffeine, paraxanthine, trigonelline, theobromine, N-acetylsphingosine, and anandamide. Therefore, it was suggested that quinic acid, caffeine, paraxanthine, trigonelline, theobromine, N-acetylsphingosine and anandamide also have a possibility to serve as some kinds of biomarkers for evaluating a ketogenic diet therapy.

<Examination Example by Another Analytical Method>

Sera collected from cancer patients before and after intake of the ketogenic diet were analyzed by an amino acid assay using HPLC and were statistically analyzed by using the resulting peak areas.

The number of the cancer patients was 13 (9 females and 4 males). As to the background of the patients, one patient had lung adenocarcinoma, one patient had ovarian cancer, one patient had nonsmall-cell lung cancer, two patients had breast cancer, one patient had left lip adenoid cystic carcinoma, one patient had peritoneal metastasis of cecal cancer, one patient had breast cancer and rectal cancer, one patient had nonsmall-cell lung cancer, one patient had bladder cancer, one patient had rectosigmoid cancer, one patient had left parapharyngeal space malignant tumor, and one patient had a metastatic pulmonary tumor.

Thirteen serum specimens collected from the cancer patients prior to intake of the ketogenic diet were analyzed by an amino acid assay using HPLC and a large number of substances in the sera were statistically analyzed. The analyzer used was L-8800 high-speed amino acid analyzer (manufactured by Hitachi, Ltd.). The result of analysis of each of the substances derived from the cancer patients were recorded as “results of analysis prior to intake of the ketogenic diet” for each patient.

Among the cases on which a consent was obtained in a clinical study conducted after obtaining the approval of the Osaka University genome review committee, the cases where patients had completed the ketogenic diet therapy for 3 months and could be clinically evaluated were adopted to the further process. The content of the ketogenic diet therapy was as described above.

Thirteen serum specimens collected from cancer patients who had taken the ketogenic diet for 3 months were analyzed by the amino acid assay using the HPLC similarly to the above, and a large number of substances in the sera were statistically analyzed. The results of analysis of each of the substances were recorded as “results of analysis after intake of the ketogenic diet” for each patient.

Complete remission group (CR group) and progressive disease/stable disease group (PD/SD group) both after intake of the ketogenic diet were as follows.

CR group: 3 patients (3 females, wherein one patient had lung adenocarcinoma, one patient had ovarian cancer and one patient had nonsmall-cell lung cancer, and they were 55.0±1.7 years old, 155±4.0 cm tall, and 46.5±2.9 kg in weight)
PD/SD group: 10 patients (6 females and 4 males, wherein two patients had breast cancer, one patient had left lip adenoid cystic carcinoma, one patient had peritoneal metastasis of cecal cancer, one patient had breast cancer and rectal cancer, one patient had nonsmall-cell lung cancer, one patient had bladder cancer, one patient had rectosigmoid cancer, one patient had left parapharyngeal space malignant tumor, and one patient had metastatic pulmonary tumor, and they were 58.5±15.5 years old, 162.1±7.7 cm tall, and 54.4±9.5 kg in weight).

The recorded “results of analysis prior to the intake of the ketogenic diet” were classified into the CR group (3 specimens) and the PD/SD group (10 specimens), and the mean value and the standard deviation of peak area were calculated for each substance. Furthermore, the peak area ratio (CR/(PD/SD)) was calculated for each substance and the significant difference was determined by the p value. Determination of the significant difference was conducted by Welch's t test. The substances found to have a significant difference between the CR group and the PD/SD group are summarized in Table 3 below together with the calculation results.

The “results of analysis after the intake of the ketogenic diet” were similarly classified into the CR group (3 specimens) and the PD/SD group (10 specimens), and the mean value and the standard deviation of peak areas were calculated for each substance. Furthermore, the peak area ratio (CR/(PD/SD)) was calculated for each substance and the significant difference was determined by the p value. The substances found to have a significant difference between the CR group and the PD/SD group are summarized in Table 4 below together with the calculation results.

TABLE 3

| Name | CR | | PD/SD | | CR/ | |
|---|---|---|---|---|---|---|
| of | Mean | Standard | Mean | Standard | (PD/ | p |
| substance | value | deviation | value | deviation | SD) | value |
| Alanine | 4.0E+02 | 3.7E+01 | 3.2E+02 | 4.7E+01 | 1.2 | 0.025 |
| Citrulline | 1.1E+01 | 3.9E+00 | 3.2E+01 | 9.1E+00 | 0.3 | 0.003 |
| Tryp- | 4.7E+01 | 7.2E+00 | 3.3E+01 | 7.1E+00 | 1.4 | 0.015 |
| tophan | | | | | | |
| Lysine | 2.4E+02 | 3.1E+01 | 1.8E+02 | 3.0E+01 | 1.4 | 0.007 |

TABLE 4

| Name | CR | | PD/SD | | CR/ | |
|---|---|---|---|---|---|---|
| of | Mean | Standard | Mean | Standard | (PD/ | p |
| substance | value | deviation | value | deviation | SD) | value |
| Cystine | 3.9E+01 | 2.0E+01 | 6.0E+01 | 9.7E+00 | 0.7 | 0.025 |
| Tyrosine | 7.2E+01 | 1.2E+01 | 5.3E+01 | 1.1E+01 | 1.3 | 0.033 |
| Histidine | 8.0E+01 | 1.0E+01 | 6.5E+01 | 9.0E+00 | 1.2 | 0.029 |

According to Table 3 above, the substances found to have a significant difference in the peak area value between the CR group and the PD/SD group prior to the intake of the ketogenic diet were alanine, citrulline, tryptophan and lysine. Alanine, citrulline, tryptophan and lysine in serum prior to intake of a ketogenic diet will serve as an indicator for determining whether the ketogenic diet therapy will be effective for a patient or not. These 4 substances are pre-intake candidate substances like the 18 substances as described above.

A cancer patient for whom a ketogenic diet therapy would be effective can be selected also by analyzing a serum derived from the cancer patient prior to intake of the ketogenic diet and using a result of analysis of at least one of alanine, citrulline, tryptophan and lysine as an indicator.

Moreover, according to Table 4 above, the substances found to have a significant difference in the peak area value between the CR group and the PD/SD group after intake of the ketogenic diet for 3 months were cystine, tyrosine and histidine. Cystine, tyrosine and histidine in serum after intake of a ketogenic diet will serve as an indicator for determining whether the ketogenic diet therapy has effectively acted or not. Histidine has been already mentioned, but the other 2 substances are also post-intake candidate substances, too.

The efficacy of a ketogenic diet therapy on a cancer patient can be predicted also by analyzing a serum derived from the cancer patient having taken the ketogenic diet and using a result of analysis of at least one of cystine and tyrosine as an indicator.

1. A method for selecting a cancer patient for whom a ketogenic diet therapy would be effective, comprising
obtaining a result of analysis of at least one pre-intake candidate substance selected from the group consisting of acylcarnitine(12:0), acylcarnitine(12:1)-2, acylcarnitine(12:1)-3, acylcarnitine(14:1)-1, acylcarnitine(14:2)-1, acylcarnitine(14:2)-2, acylcarnitine(14:3)-2, acylcarnitine(16:1), acylcarnitine(16:2), anandamide, heptadecenoic acid, hypotaurine, linoleylethanolamide, palmitoleic acid, palmitoylcarnitine, phytosphingosine, quinic acid, uric acid, alanine, citrulline, tryptophan and lysine in a serum derived from a cancer patient prior to intake of a ketogenic diet, and using the result of analysis as an indicator.

2. A method for predicting efficacy of a ketogenic diet therapy in a cancer patient, comprising
obtaining a result of analysis of at least one post-intake candidate substance selected from the group consisting of diethanolamine, histidine, hydroxyproline, stearoylethanolamide, stigmasterol, cystine and tyrosine in a serum derived from a cancer patient having taken a ketogenic diet, and using the result of analysis as an indicator.
